(19) United States
(12) Patent Application Publication
(10) Pub. No.: US 2010/0071707 A1
(43) Pub. Date: Mar. 25, 2010

(54) External middle ear insufflation device

(76) Inventor: Daniel L. Wohl, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/586,318

(22) Filed: Sep. 21, 2009

Related U.S. Application Data
(60) Provisional application No. 61/192,756, filed on Sep. 22, 2008.

(51) Int. Cl. A61F 11/08

(52) U.S. Cl. 128/864

(57) ABSTRACT

A middle ear insufflation device to relieve pressure differential across the tympanic membrane resulting from rapid changes in altitude or intrinsic Eustachian Tube Dysfunction, the device having a compressible bladder member and an ear canal sealing member such that the bladder member can be compressed and released to reduce or increase pressure within the ear canal that is then translated across the tympanic membrane affecting internal pressure within the middle ear space, thereby helping to facilitate normalization of middle ear pressure through the Eustachian Tube.

This application claims the benefit of U.S. Provisional Patent Application Ser. No. 61/192,756, filed Sep. 22, 2008.

### BACKGROUND OF THE INVENTION

This invention relates generally to the field of devices and methods used to equalize air pressure differentials between the middle ear and the external ear canal resulting from rapid changes in altitude, such as occur during take off and landing of an airplane, or with primary Eustachian Tube Dysfunction as is commonly encountered in children.

As an airplane rises to cruising altitude the air pressure within the ear canal, the region exterior to the tympanic membrane, drops rapidly, while the air pressure within the middle ear remains higher unless equalization occurs. During landing the opposite situation occurs, with the external air pressure now being greater than the internal air pressure. In most cases, sufficient air passes from inside the upper throat through the Eustachian Tubes into the middle ear to equalize the air pressure within a brief amount of time. In other cases, however, the Eustachian Tubes may not open sufficiently or may be blocked, as for example when a person is suffering from a cold or with young children whose Eustachian Tubes have not fully matured, with the pressure differential across the tympanic membrane resulting in discomfort or pain. Yawning, chewing gum, or “popping” the ears by closing the nostrils and mouth while expelling air from the lungs are techniques often utilized. These techniques do not always work, and in the case of infants and young children, the child may not understand the parent's instructions.

Devices and techniques addressing this problem in the past have usually been based on the Politzer method, wherein air is forced through the nasal cavity. The original method required a high pressure burst administered simultaneously with swallowing, which proved difficult to properly time. In addition, the early systems lacked safeguards against the administration of excessive pressure. The devices were often designed only to increase pressure within the Eustachian tube internal to the middle ear, and thus were not useful in circumstances where the external pressure is less than the internal pressure. Even with improved devices, alleviating the discomfort and pain in young children and infants was hindered by the natural aversion to having air forced into the nasal cavity.

Thus there is a need for a simple device and method that can be utilized to quickly alleviate discomfort and pain caused by unequalized, sometimes rapid, air pressure differentials encountered during both take-off and landing, as well as in instances of primary Eustachian Tube Dysfunction or other conditions resulting in discomfort or pain. There is a further need for a relatively simple device and method that can be easily administered to young children and infants, and one that does not present a danger of excessive pressure or suction.

### SUMMARY OF THE INVENTION

The invention is an external middle ear insufflation device that rapidly equalizes pressure differentials between the external ear canal and the middle ear to alleviate the resulting discomfort and pain, and its method of use. The device comprises in general a compressible bladder member of relatively small volume, the bladder member having a main body and a single opening for suction and expulsion of air into and from the interior of the bladder member. An ear canal sealing member, that may be mounted onto or connected to the bladder member or alternatively formed as a unitary body with the bladder member, forms a seal against the ear canal of the person being treated. The ear canal sealing member comprises an internal bore in communication with the opening of the bladder member, and may further comprise an annular sealing portion of relatively large diameter that transitions into a smaller end member.

When air pressure differentials are encountered, the ear canal sealing member of the device is inserted into the ear canal so as to effectively block passage of air between the ear canal and the environment. When the external air pressure is greater than the air pressure in the middle ear, the bladder member is first squeezed to expel air within its interior, the ear canal sealing member is then inserted into the ear canal, and the bladder member is allowed to expand to pull air from the ear canal into the bladder member, thereby decreasing pressure within the ear canal, which is translated to the middle ear as the tympanic membrane responds by moving in a normal piston-like manner resulting in corresponding changes in the middle ear pressure. When the external air pressure is less than the air pressure in the middle ear, the bladder member is inserted in its expanded state and then squeezed using the fingers and thumb in order to increase pressure in the ear canal against tympanic membrane. In certain circumstances, repetitive pumping of the device by squeezing and releasing the bladder member may be needed to equalize the atmospheric air pressure differential by promoting air entry into the middle ear through the Eustachian Tube as the tympanic membrane moves back and forth, thereby relieving the pain and discomfort from pressure imbalance.

### BRIEF DESCRIPTION OF THE DRAWINGS

FIG. 1 is a view of an embodiment showing the compressible bladder member and the ear canal sealing member.

FIG. 2 is a cross-sectional view of the embodiment of FIG. 1 showing the compressible bladder member and the ear canal sealing member.

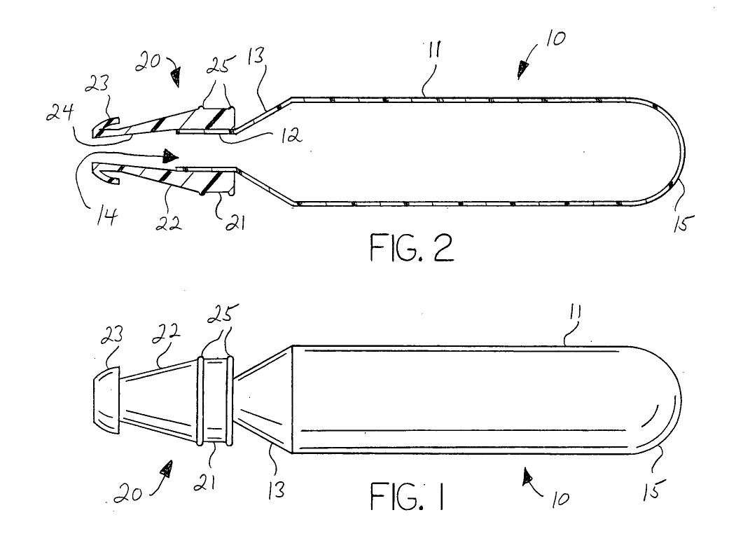

### DETAILED DESCRIPTION OF THE INVENTION

With reference to the drawings, the invention will now be described in detail with regard for the best mode and the preferred embodiment. In general, the invention is an external middle ear insufflation device and its method of use that rapidly equalizes pressure differentials between the external ear canal and the middle ear, such as would be encountered for example during the take off and landing phases of air travel, or with certain medical conditions. The device allows an individual to address pain and discomfort in his or her own ears, as well as addressing pain and discomfort in the ears of an infant or young child incapable of operating the device. The device transfers a relatively small, controlled amount of air within the ear canal during suction or expulsion operations.

As shown in the figures illustrating a representative embodiment of the invention, the middle ear insufflation device comprises a compressible bladder member 10 and an ear canal sealing member 20. The bladder member 10 and ear canal sealing member 20 may be formed as a unitary member, but preferably comprise separate elements with the ear canal sealing member 20 mounted onto or connected to the bladder member 10. The bladder member 10 is a thin-walled member composed of a relatively soft plastic, but the walls must be of sufficient rigidity that the bladder member 10 maintains an expanded configuration defining an interior volume when the bladder member 10 is not placed under compressive force. i.e., when the bladder member 10 is in a neutral status. Preferably the bladder member comprises a generally elongated main body 11 with a closed end 15, the other end of the main body comprising a short tube member 12 defining a single opening 14 into the interior of the main body 11, the opening 14 being of relatively small diameter. A transition shoulder 13 may be provided between the main body 11 and the tube member 12.

The ear canal sealing member 20 is preferably composed of a relatively soft and flexible plastic, and in the embodiment shown comprises an annular sealing portion 21 sized to seal off the ear canal such that air cannot pass between the ear canal and the environment when the device is in use. The sealing portion 21 may be provided with one or more annular rib members 25 to more effectively seal the ear canal. A generally conical shaped transition portion 22 extends outwardly from the sealing portion 21, and an internal bore 24 receives and communicates with the tube member 12 of the bladder member 10 such that air can be transferred into and out of the device. A cushioning end member 23 may be positioned at the free end of the transition portion 22. Alternative constructions for the ear canal sealing member 20 may comprise similar structures that effectively seal the external ear canal.

In the embodiment wherein the ear canal sealing member 20 is a separate member joined to the bladder member 10, such as shown in the figures with the annular sealing portion 21 of the ear canal sealing member 20 mounted onto the tube member 12 of the bladder member 10, varying sizes of ear canal sealing members 20 can be chosen as needed, to best fit the size of the external auditory canal, or if there is need for the ear canal sealing member 20 to be replaced if it becomes damaged or soiled.

Without the intention of being limited to the dimensions as now set forth, suitable representative dimensions for the middle ear insufflation device may consist of, with all dimensions being approximations, a bladder member 10 having a main body 11 thirteen mm in diameter and four cm in length, a shoulder 13 eight mm in length, and a tube member 12 eight mm in length and five mm in diameter. The ear canal sealing member 20 may be seventeen mm in length and have a sealing portion 21 with a diameter of one cm and a length of three mm, a transition portion one cm in length and an end member 23 three mm in length. The volume of the bladder member 10 is approximately 5 milliliters, but may be slightly larger or smaller provided the characteristics of the device are not excessively altered.

The method of using the middle ear insufflation device is straightforward. When middle ear—tympanic membrane air pressure differentials are encountered, the ear canal sealing member 20 inserted into the ear canal to the point where the annular sealing portion 21 effectively blocks passage of air between the ear canal and the environment creates a controlled system. When the external air pressure is less than the air pressure in the middle ear, the bladder member 10 is then squeezed using the fingers and thumb in order to increase pressure in the ear canal against tympanic membrane. When the external air pressure is greater than the air pressure in the middle ear, the bladder member 10 is first compressed to expel air within its interior, the ear canal sealing member 20 is then inserted into the ear canal, and the bladder member 10 is allowed to expand to pull air from the ear canal into the bladder member 10, thereby decreasing pressure within the ear canal. In certain circumstances repetitive pumping by compressing and releasing the bladder member 10 may be needed to equalize the middle ear air pressure differential to relieve the pain and discomfort. This pumping action results in normal repetitive piston-like movement of the tympanic membrane and assists in the transfer of air through the Eustachian Tube into the middle ear, thereby normalizing the pressure differential across the tympanic membrane. Because of the relatively small volume of the bladder member 10, relatively small changes in pressure on the order of 50 to 400 mm of water are achieved during usage, thereby insuring that suction or increased pressure on the tympanic membrane is not harmful.

It is understood that equivalents and substitutions for certain elements set forth above may be obvious to those of ordinary skill in the art, and thus the true scope and definition for the invention is to be as set forth in the following claims.

I claim:

1. A method of reducing an air pressure differential between the external ear canal and the internal middle ear, the method comprising the steps of:
providing an insufflation device comprising a compressible bladder member and an ear canal sealing member, said device adapted to expel or suction air and structured so as to be insertable into an external ear canal;
ascertaining whether the air pressure differential is such that the external pressure is greater than the pressure within the middle ear or whether the external pressure is less than the pressure within the middle ear;
if the external pressure is greater than the pressure within the middle ear, compressing said bladder member to expel air within the bladder member, inserting said insufflation device into the external ear canal such that said ear canal sealing member seals the ear canal, and releasing the compression of said bladder member to suction air from the ear canal into said bladder member; and
if the external pressure is less than the pressure within the middle ear, inserting said insufflation device into the external ear canal such that said ear canal sealing member seals the ear canal, compressing said bladder member to expel air from said bladder member into the ear canal.

2. The method of claim 1, further comprising the step of repetitively compressing and releasing said bladder member.

3. The method of claim 1, wherein said step of expulsing or suctioning air is limited to transferring a maximum approximately 5 milliliters of air.

4. The method of claim 1, wherein said step of expulsing or suctioning air produces a pressure of approximately 50 to 400 mm of water.

5. The method of claim 2, wherein said step of expulsing or suctioning air is limited to transferring a maximum approximately 5 milliliters of air.

6. The method of claim 2, wherein said step of expulsing or suctioning air produces a pressure of approximately 50 to 400 mm of water.

7. The method of claim 3, wherein said step of expulsing or suctioning air produces a pressure of approximately 50 to 400 mm of water.

8. The method of claim 5, wherein said step of expulsing or suctioning air produces a pressure of approximately 50 to 400 mm of water.

9. An ear insufflation device capable of reducing an air pressure differential between the external ear canal and the internal middle ear, said device comprising:
a compressible bladder member adapted to expel and suction air, said compressible bladder member having a volume of approximately 5 milliliters; and
an ear canal sealing member joined to said compressible bladder member and adapted to seal an external ear canal upon insertion into the external ear canal.

10. The ear insufflation device of claim 9, wherein said compressible bladder member is thin-walled and composed of plastic; and
wherein said ear canal sealing member comprises an annular sealing portion and is composed of plastic.

11. The device of claim 10, wherein said annular sealing portion comprises one or more annular rib members.

12. The device of claim 10, said ear canal sealing member further comprising a cushioning end member.

13. The device of claim 11, said ear canal sealing member further comprising a cushioning end member.

14. The device of claim 9, wherein said compressible bladder member and said ear canal sealing member are separable.

15. The device of claim 9, wherein said compressible bladder member produces a pressure of approximately 50 to 400 mm of water.

16. An ear insufflation device capable of alleviating pain and discomfort arising from an air pressure differential between the external ear canal and the internal middle ear, said device comprising:
a compressible, thin-walled, bladder member adapted to expel and suction air, said compressible bladder member, said compressible bladder member comprising an elongated tube member; and
an ear canal sealing member joined to said compressible bladder member and adapted to seal an external ear canal upon insertion into the external ear canal, said ear canal sealing member comprising a cushioning member, an annular sealing portion having one or more annular rib members, and a bore receiving said tube member.

17. The device of claim 16, wherein said compressible bladder member and said ear canal sealing member are separable.

18. The device of claim 16, said compressible bladder member having a volume of approximately 5 milliliters.

19. The device of claim 16, wherein said compressible bladder member produces a pressure of approximately 50 to 400 mm of water.
